(19) United States
(12) Patent Application Publication
(10) Pub. No.: US 2024/0374265 A1
(43) Pub. Date: Nov. 14, 2024

(54) ORTHOPEDIC DRILL GUIDE

(71) Applicant: DePuy Synthes Products, Inc., Raynham, MA (US)

(72) Inventors: Michael MCGURK, Williamstown, NJ (US); Jason LONG, West Chester, OH (US); John HUNT, Blue Ash, OH (US)

(21) Appl. No.: 18/653,236

(22) Filed: May 2, 2024

Related U.S. Application Data
(60) Provisional application No. 63/500,047, filed on May 4, 2023.

(51) Int. Cl. A61B 17/17; A61B 17/00

(52) U.S. Cl. CPC A61B 17/1728; A61B 2017/00469

(57) ABSTRACT

An ergonomic drill guide device with increased visibility is provided. The drill guide device has a handle with a top and bottom. The handle includes a rod and a hand grip. The rod has an outer dimension R. The hand grip at least partially surrounds the rod and has an end portion. The hand grip has an outer dimension H at an end. The drill guide has an outer dimension D. There is a gap G between the end of the end portion and the drill guide. The ratio of R to D is between 0.2 to 1.0.

### CROSS-REFERENCE TO RELATED APPLICATIONS

The present application claims priority of U.S. Provisional Patent Application Ser. No. 63/500,047 filed May 4, 2023, with the U.S. Patent & Trademark Office, the contents of which are hereby incorporated by reference in their entirety.

### TECHNICAL FIELD

Various exemplary embodiments disclosed herein relate generally to drill guides that allows surgeons to drill holes for use with a bone plate and its corresponding bone screws.

### BACKGROUND

Bone plates are widely used to secure bone fractures. Such bone plates include screw holes. Screws are inserted through the holes and into the fractured bone. Often, a surgeon will use a drill guide to accurately drill holes into the bone so that the screws end up at a desired angle or trajectory (e.g., passing through desired bone portions). The drill guide ensures that the hole is aligned at the desired angle or trajectory. Some screws are inserted at an angle that is aligned with the center axis of the hole (e.g., “standard” or lagging screws). Other screws, variable angle screws, are inserted at an angle to the axis of the hole. Variable angle screws are typically designed for insertion of the screw at angles up to 15 degrees from the center axis of the hole, for example.

Variable angle screws provide the ability to create a bone plate construct while also allowing the surgeon the freedom to choose the screw trajectory. With variable angle screw technology, screw angulation is free to vary within a specified cone angle around the central axis of the plate hole. Variable screw positioning offers many benefits by allowing the surgeon to: target the screw into high-quality bone; adjust screw direction after bending the plate; position screws precisely to avoid joint penetration; redirect screw position to avoid existing implants or other screws; and adapt screw position to accommodate varied patient anatomy and capture fracture fragments.

Existing drill guides do not provide optimal usefulness.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

In one embodiment, a drill guide device, comprises a handle extending along a longitudinal handle axis. The handle has a top and a bottom. The handle comprises a rod extending along the longitudinal axis. The rod has a top and a bottom. The rod has an outer dimension R as viewed from the top of the handle. The handle includes a hand grip extending along the longitudinal handle axis and at least partially surrounds the rod. The hand grip has a first end portion along the longitudinal handle axis, a second end portion opposite the first end portion. The hand grip has an outer dimension H at the first end portion as viewed from the top of the handle. The drill guide device includes a drill guide extending along a longitudinal drill guide axis and forming a hollow channel configured and adapted to receive a drill bit. The drill guide has an outer dimension D as viewed from the top of the handle. The drill guide comprises a proximal end having an opening to the hollow channel, a distal end having an opening to the hollow channel and the drill guide is coupled to the rod at an angle. There is a gap having a dimension G between the end of first end portion and drill guide measured along the bottom of rod. The outer dimension R of the rod is measured proximate the drill guide and the ratio of R to D is between 0.2 to 1.0.

### BRIEF DESCRIPTION OF THE DRAWINGS

In order to better understand various exemplary embodiments, reference is made to the accompanying drawings, wherein:

FIG. 1 illustrates a perspective view of one exemplary embodiment of a drill guide device; and

FIG. 2 illustrates a side view of the exemplary embodiment of the drill guide device of FIG. 1;

FIG. 3 illustrates a top view of the exemplary embodiment of the drill guide device of FIG. 1;

FIG. 4 illustrates a perspective view of a drill guide device from the top position;

FIG. 5 illustrates a top plan view of the drill guide device of FIG. 4;

FIG. 6 illustrates a bottom plan view of the drill guide device of FIG. 4;

FIG. 7 illustrates a left side view of the drill guide device of FIG. 4;

FIG. 8 illustrates a right side view of the drill guide device of FIG. 4;

FIG. 9 illustrates a front elevation view of the drill guide device of FIG. 4;

FIG. 10 illustrates a back elevation view of the drill guide device of FIG. 4;

FIG. 11 illustrates a perspective view of a drill guide device from the top position;

FIG. 12 illustrates a top plan view of the drill guide device of FIG. 11;

FIG. 13 illustrates a bottom plan view of the drill guide device of FIG. 11;

FIG. 14 illustrates a left side view of the drill guide device of FIG. 11;

FIG. 15 illustrates a right side view of the drill guide device of FIG. 11;

FIG. 16 illustrates a front elevation view of the drill guide device of FIG. 11;

FIG. 17 illustrates a back elevation view of the drill guide device of FIG. 11;

FIG. 18 illustrates a perspective view of a drill guide device from the top position;

FIG. 19 illustrates a top plan view of the drill guide device of FIG. 18;

FIG. 20 illustrates a bottom plan view of the drill guide device of FIG. 18;

FIG. 21 illustrates a left side view of the drill guide device of FIG. 18;

FIG. 22 illustrates a right side view of the drill guide device of FIG. 18;

FIG. 23 illustrates a front elevation view of the drill guide device of FIG. 18;

FIG. 24 illustrates a back elevation view of the drill guide device of FIG. 18;

To facilitate understanding, identical or similar reference numerals have been used to designate elements having substantially the same or similar structure and/or substantially the same or similar function.

### DETAILED DESCRIPTION

The description and drawings illustrate the principles of the present disclosure. It will thus be appreciated that those skilled in the art will be able to devise various arrangements that, although not explicitly described or shown herein, embody the principles herein. Furthermore, all examples recited herein are principally intended expressly to be for pedagogical purposes to aid the reader in understanding the principles of the present disclosure and the concepts contributed to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

As used herein, any reference to “some implementations,” “an embodiment,” “some embodiments,” “one example,” “for example,” or “an example” means that a particular element, feature, structure, or characteristic described in connection with the embodiment is included in at least some implementations. The appearance of the phrase “in some embodiments” or “one example” in various places in the specification is not necessarily all referring to the same embodiment, for example. Further, all references to one or more embodiments or examples are to be construed as non-limiting to the claims. Also, reference numerals, if included in the claims, are not intended to limit the claims to the specific embodiments in the figures.

The illustrations are generally described below as they appear in the figure itself. Such description should not be interpreted as a limitation of the invention, but merely as a description of the figure. That is, if the description of FIG. A notes that element X is connected to element Y, this is merely a description of the embodiment shown in the figure and it should not be construed as a narrowing or limitation of the invention to require that element X always be connected to element Y (or even that element X or element Y are required components).

Bone plates may be used to repair a bone fracture by securing two bone fragments together while the bone heals itself naturally. The bone plate is typically shaped in a complementary manner to the bone so it can span the length of the bone and across the fracture site. The bone plate includes holes at several locations along its length (and sometimes along its width) to allow bone screws to be placed through the holes and into the bone, thereby creating a stable construct to facilitate the natural healing process. The screws secure the plate to the bone on both sides of the fracture. Ideally, the bone fragments will also be pulled tightly together which is salubrious for the healing process. This “pulling together” is often referred to as compression or fracture reduction.

Plates have been designed to facilitate such fracture reduction and are sometimes referred to as compression plates. Compression plates are designed to cooperate with screws by having a hole opening with a size that is oversized from the screw shaft size. This provides a gap between the screw shaft and the screw hole. Placing the screw in the hole with the gap between the screw and the hole wall in a desired offset and orientation (e.g., offset from the center by 1 mm and offset towards the left side of the hole) can produce a lateral force on the plate during insertion of the screw (i.e., when the screw head contacts with hole wall during insertion). For example, if a screw with a sloped head is used and positioned on the left side of the screw hole, during insertion the sloped portion of the screw head contacts the left side of the hole which produces a leftward force on the plate as the screw continues to be advanced and the sloped head of the screw presses against the hole wall.

This lateral force can be used to the advantage of the surgeon and to the benefit of the patient. To implement compression, the bone plate is placed along the bone and across the fracture site. A first screw is placed in a first hole securing the plate to the first bone fragment. The first screw is tightened securing the first screw and the plate to the first bone fragment. Next, a second screw is inserted through a second plate hole (on an opposite side of the fracture from the first screw hole) and into the second bone fragment. Now the two bone fragments can be drawn together in compression. The second screw is placed in the second plate hole and is advantageously placed offset from the center of the second plate hole whereby the second screw head will interfere with the side of the second hole during insertion. As the second screw is inserted, it contacts the side wall of the second screw hole and pushes the bone laterally (as the second screw moves from its offset position towards the center of the hole (which naturally occurs as the sloped screw head moves into the screw hole and begins to contact the wall of the second screw hole). This moves the second bone fragment closer to the first bone fragment (reduction) at the same time that the whole construct is being tightened up.

Because fracture reduction and compression can be a finicky process (the screws and holes can be quite small the desired gaps even smaller), drill guides are often used to predrill the holes for the bone screws making the process more accurate. The drill guide may help the surgeon offset the drill hole from the center of the plate hole to facilitate this reduction and compression. In the case of drilling an offset hole, the tip of the drill guide may be undersized from the plate hole giving the surgeon the option to place drill a hole in various offset locations and orientations relative to the plate hole. In other situations, the hole may be drilled in the center of the plate hole. In the case of drilling a centered hole, the tip of the drill guide may have a rounded portion to position or “automatically center” the drill guide to the center of the plate hole.

Many screw holes are relatively small. Getting the desired offset gap and angle correct can be challenging especially with views obstructed by tissue, retractors, and large bulky instruments. Small instruments may be tough to position accurately because they may be difficult to properly grip. Getting the drill guide tip appropriately into the desired portion relative to the hole to create reduction and compression can also be challenging. A drill guide that is big and bulky can obstruct the view of the hole and make drill guide tip placement challenging.

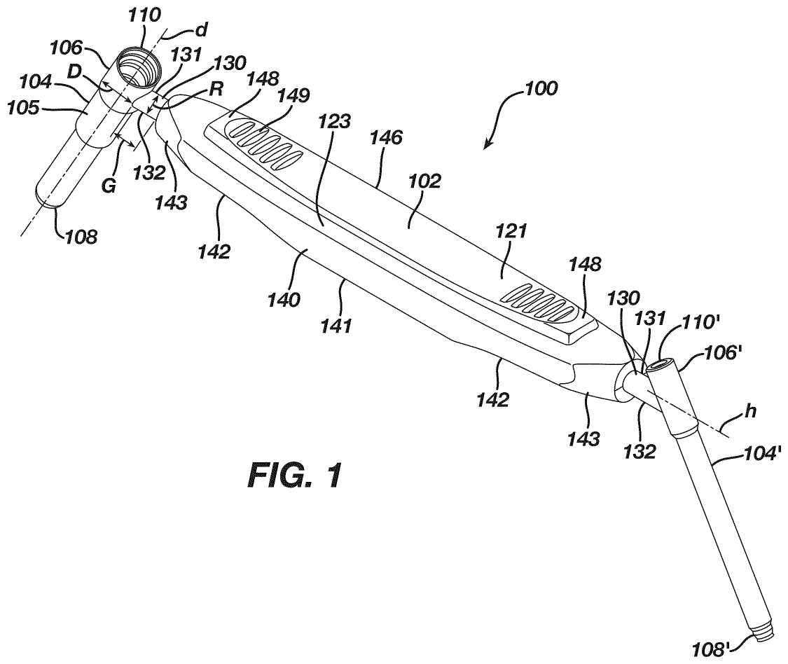

FIG. 1 illustrates a perspective view of one exemplary embodiment of a drill guide device. As shown, drill guide device 100 includes a drill guide handle 102. Handle 102 extends longitudinally along a longitudinal handle axis h. Handle 102 has a top 121, a bottom 122 generally opposite the top 121 and sides 123 that run from the top 121 to the bottom 122. Handle 102 is configured and shaped to be grasped by a user, described in more detail below.

Handle 102 includes a central rod 130 that is cylindrical and extends along the longitudinal handle axis h of the handle 102. Rod 130 has a top 131 and a bottom 132 generally opposite the top 131. Rod 130 is typically metal but could be made of other materials that provide strength. Rod 130 is cylindrically shaped and has a diameter of R; however, rod 130 can have other cross-sectional shapes. The relevant measure of R in the case of a cylindrical rod is the diameter of rod 130 viewed from the top of drill guide device 100 because this is a common view for the user when the drill guide 100 is in use. If rod 130 is implemented with a square cross section, instead of diameter R, the relevant measure would be the width R′ (not shown) of rod 130, viewed from the top of drill guide device 100. The diameter and width are collectively referred to as outer dimension. Outer dimension is intended to apply to any shape cross-section and refers to the dimension of the rod (or other element, e.g., hand grip 140 or drill guide 104 described in more detail below) that is obstructing the surgeon's view of the plate holes as viewed from the top of drill guide device 100.

Rod 130 is covered with hand grip 140. Hand grip 140 does not extend all the way to the ends of rod 130. This might advantageously help the surgeon's visibility near where the holes are to be drilled and the screws to be placed (described in more detail below). Hand grip 140 is typically hard plastic but could be other materials. The plastic provides a structure that can be gripped, but without adding unnecessary weight which might fatigue a user over time. Alternatively, hand grip 140 and rod 130 may be one monolithic part if sufficient strength and weight can be provided.

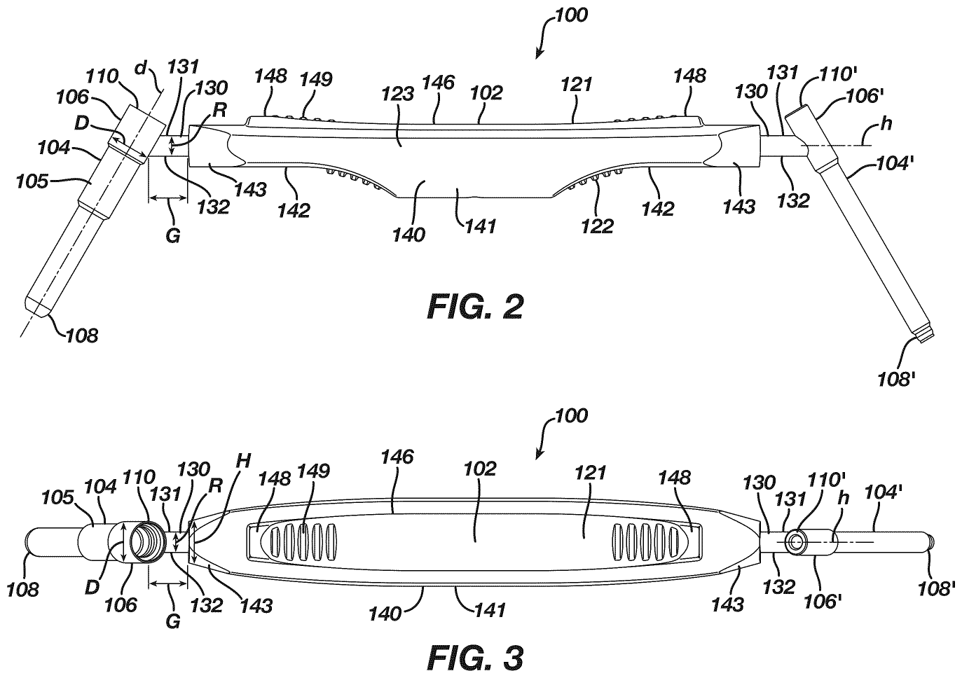

Hand grip 140 has a thicker middle portion 141, thinner lateral portions 142, and even thinner end portions 143. Hand grip 140 can be any shape that can be gripped by a surgeon's hand. Hand grip 140 has a chamfered square cross section at the end portion 143 with a width of H at the end of end portion 143, as viewed from the top of drill guide device 100 (best seen in FIG. 2). This might also advantageously help the surgeon's visibility near where the holes are to be drilled. Hand grip 140 can have other cross-sectional shapes at the end portion 143. If hand grip 140 has a round cross section at the end portion 143, instead of a chamfered square, the relevant measure would be the diameter H′ (not shown) of hand grip 140, viewed from the top of drill guide device 100. The diameter and width are collectively referred to as outer dimension. Outer dimension is intended to apply to any shape cross-section and refers to the dimension of the hand grip 140 that is obstructing the surgeon's view of the plate holes as viewed from the top of drill guide device 100.

Hand grip 140 further includes a top grip 146 disposed on the top of hand grip 140. Top grip 140 can be made of the same material as hand grip 140 or may be made of a softer material for ergonomic purposes. Top grip 146 extends along the top of hand grip 140. Top grip 146 includes raised portions 148 on opposite sides of top grip 146. Raised portion 148 include ridges 149 disposed on top of each raised portion 148 and oriented generally perpendicular to the longitudinal axis h of handle 102. Ridges 149 and raised portions 148 may provide a comfortable thumb rest with increased friction (ridges) for the surgeon to control drill guide device 100.

Drill guide assembly 100 further includes a drill guide 104. Drill guide 104 is a generally tubular structure that defines a drill guide hollow channel 110 that extends along a longitudinal drill guide axis d. Drill guide 104 has a proximal end 106 and a distal end 108 opposite the proximal end 106. Drill guide hollow channel 110 is configured and adapted to accept and guide drill bits (not shown) used to drill a hole in the bone. Drill guide 104 also has a middle portion 105 between proximal end 106 and a distal end 108. The diameter of drill guide 104 at proximal end 106, at the location where the bottom 132 of rod 130 couples to drill guide 104 has an outer diameter of D. Drill guide 104 which lies along drill guide axis d couples to rod 130 which lies along longitudinal handle axis h at an angle which is an obtuse angle.

The middle portion 105 of drill guide 104 has a smaller outer diameter than the outer diameter D. The proximal end 106 of drill guide 104 has a larger outer diameter than the outer diameter of the distal end 108 of drill guide 104. The proximal end 106 of drill guide 104 may be sized and configured to receive a spring-loaded device (not shown) such as described in U.S. patent application Ser. No. 18/052,480. Drill guide 104 may alternatively have a single outer diameter from the proximal end 106 to the distal end 108, or may have two different outer diameters, or any number of outer diameters.

Distal end 108 may have teeth on its edge which facilitate a more stable contact with the bone during drilling operations. Although not shown, distal end 108 may have a partially rounded end for mating with a screw hole (e.g., for a screw that is desired to be centered, not offset from the hole axis).

As shown in FIGS. 1-3, drill guide handle 102 is attached to two different drill guides 104 and 104′ at opposite portions of the drill guide handle 102. Drill guides 104, 104′ may have different characteristics for use in different situations like different size screws or tighter access to the bone.

As shown in FIG. 1, drill guide device 100 further include a second drill guide 104′. Drill guide 104′ is a generally tubular structure that defines a drill guide hollow channel 110′ that extends along a longitudinal drill guide axis d′ (not shown). Drill guide 104′ has a proximal end 106′ and a distal end 108′ opposite the proximal end 106′. Drill guide hollow channel 110′ is configured and adapted to accept and guide drill bits (not shown) used to drill a hole in the bone. Drill guide 104′ has a proximal end 106′ and a distal end 108′. The diameter of drill guide 104′ at proximal end 106′, at the location where the bottom 132 of rod 130 couples to drill guide 104′ has an outer diameter of D′ (not shown).

The hollow channels 110, 110′ can be concentric with the drill guide 104, 104′ or can alternatively be offset within the drill guide 104, 104′ to provide for a predetermined offset drill hole with respect to the plate hole.

Drill guide device 100 may be used in combination with various designs of drill guide sleeves (not shown) which may be placed in drill guide 104 to help center an undersized drill bit in drill guide hollow channel 110. Drill guide sleeves may also protrude past distal end 108 to provide different interfaces to the bone/plate area (e.g., partially rounded drill guide sleeve, jagged drill guide sleeve, threaded to mate to bone hole threads,). Various mating mechanisms may be used between drill guide 104 and the drill guide sleeves (e.g., sliding, threaded, etc.). The dimensions of a drill guide sleeve may be selected to extend various distances from distal end 108 to accommodate different plate thicknesses as well as to allow for offset drilling (i.e., a drill location with an offset gap between the threaded hole axis and the drill bit or screw axis). As a result, the drill guide sleeve may protect the bone plate from the drill bit no matter the thickness of the bone plate.

If an angled screw hole is to be drilled, the surgeon may tilt the drill guide to the desired angle to drill the hole at the desired angle to drill the hole. Next, the surgeon inserts a drill bit (not shown) into the drill guide 104 and drills the hole.

The surgeon may also drill offset holes for compression. In this situation, distal end 108 may be placed in the plate hole in an offset position (i.e., the center of the distal end being offset from the center of the screw hole). At this point, the surgeon inserts the drill into the drill guide to drill the hole.

The dimensions and materials of drill guide device 100 are designed for ergonomics and increased visibility.

As noted above, drill guide device 100 includes a drill guide 104. Drill guide 104 is secured to rod 130 at an angle. Drill guide 104 can be secured to rod 130 via welding or other techniques. Drill guide 104 is cylindrically shaped has an outer diameter of D along bottom 132 of rod 130 where drill guide 104 is secured to rod 130. Drill guide 104 can have other cross-sectional shapes. If drill guide 104 has an outer square cross section, instead of diameter D, the relevant measure would be the width D′ (not shown) of drill guide 104, viewed from the top of drill guide device 100. The diameter and width are collectively referred to as outer dimension. Outer dimension is intended to apply to any shape cross-section and refers to the dimension of the drill guide 104 that is obstructing the surgeon's view of the plate holes as viewed from the top of drill guide device 100 and at the bottom 132 of rod 130 where drill guide 104 is secured to rod 130.

One distal end 108 may have teeth while the other distal end 108 at the opposite side of drill guide handle 102 may have a rounded end or any such combination. Further, each distal end 108 may have the same type of end. Also, drill guide device 100 may be a single-sided drill guide device with only one drill guide 104 or 104′ provided.

As noted above, hand grip 142 does not extend all the way to the ends of rod 130 to help the surgeon's visibility of distal end 108 while drilling holes into the bone. The distance between the end of end portion 143 and drill guide 104, measured along the bottom 132 of rod 130 is gap G. Gap G provides visibility as noted above, however, if gap G is too large then the surgeon's control and tactile feedback becomes reduced. Also, if H is too small, the surgeon's control and tactile feedback becomes reduced.

The dimensions D, R, G, and H, and their ratios, are designed to help the surgeon's visibility of distal end 108 while drilling holes into bone. If the surgeon has improved visibility of distal end 108, it might be easier to accurately drill the holes used for reduction and compression.

In the case of drill guide 104, the drill guide 104 has a larger outer diameter D to accommodate an internal sleeve insert, for example, possibly with a spring loading. In the case of drill guide 104, D is 8 mm, R is 4 mm, H is 8 mm, and G is 4 mm. In the case of drill guide 104, the ratio of R/D is 4/8 or 0.5; the ratio of H/R is 8/5 or 1.6; the ratio of D/G is 8/4 or 2.0.

In a preferred alternate to the case of drill guide 104, the ratio of R/D ranges from 0.4 to 0.6, the ratio of H/R ranges from 1.5 to 1.7, and the ratio of D/G ranges from 1.5 to 2.5. In another preferred alternate to the case of drill guide 104, the ratio of R/D ranges from 0.3 to 0.7, the ratio of H/R ranges from 1.3 to 1.9, and the ratio of D/G ranges from 1.0 to 5.0. In another preferred alternate to the case of drill guide 104, the ratio of R/D ranges from 0.2 to 1.0, the ratio of H/R ranges from 1.0 to 2.0, and the ratio of D/G ranges from 0.1 to 10.0.

In the case of drill guide 104′, D is 5 mm, R is 4 mm, H is 8 mm, and G is 4 mm. In the case of drill guide 104′, the ratio of R/D is 4/5 or 0.8 (smaller provides more visibility but less strength); the ratio of H/R is 8/5 or 1.6 (smaller provides better visibility but might be less ergonomic); the ratio of D/G is 5/4 or 1.25 (smaller provided better visibility but can provide less tactile feedback to the surgeon).

In a preferred alternate to the case of drill guide 104′, the ratio of R/D ranges from 0.7 to 0.9, the ratio of H/R ranges from 1.5 to 1.7, and the ratio of D/G ranges from 1.2 to 1.3. In another preferred alternate to the case of drill guide 104′, the ratio of R/D ranges from 0.6 to 1.0, the ratio of H/R ranges from 1.3 to 1.9, and the ratio of D/G ranges from 1.0 to 2.0. In another preferred alternate to the case of drill guide 104′, the ratio of R/D ranges from 0.4 to 1.0, the ratio of H/R ranges from 1.0 to 2.0, and the ratio of D/G ranges from 0.1 to 10.0.

In another preferable embodiment, the ratio of R/D ranges from 0.4 to 0.9, the ratio of H/R ranges from 1.5 to 2.0, and the ratio of D/G ranges from 1.2 to 2.1. In another preferable embodiment, the ratio of R/D ranges from 0.4 to 1.0, the ratio of H/R ranges from 1.0 to 4.0, and the ratio of D/G ranges from 0.5 to 5.0. In another preferable embodiment, the ratio of R/D ranges from 0.4 to 1.0, the ratio of H/R ranges from 1.0 to 8.0, and the ratio of D/G ranges from 0.1 to 10.0.

Various embodiments of drill guides have been described. These drill guides allow a surgeon to drill offset holes for compression places. Further, they allow the surgeon to drill neutral holes or angled neutral holes.

While each of the embodiments are described above in terms of their structural arrangements, it should be appreciated that the invention also covers the associated methods of using the embodiments described above.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications and combinations of the various embodiments can be affected while remaining within the spirit and scope of the invention. Accordingly, the foregoing disclosure, description, and figures are for illustrative purposes only and do not in any way limit the invention, which is defined only by the claims.

What is claimed is:

1. A drill guide device, comprising:
a handle extending along a longitudinal handle axis (h), the handle having a top and a bottom, the handle comprising:
a rod extending along the longitudinal axis (h), the rod having a top and a bottom, the rod having an outer dimension R as viewed from the top of the handle; and
a hand grip extending along the longitudinal handle axis (h) and at least partially surrounding the rod, the hand grip having a first end portion along the longitudinal handle axis (h), a second end portion opposite the first end portion, the hand grip having an outer dimension H at the first end portion as viewed from the top of the handle; and
a drill guide extending along a longitudinal drill guide axis (d) and forming a hollow channel configured and adapted to receive a drill bit, the drill guide having an outer dimension D as viewed from the top of the handle, the drill guide comprising:
a proximal end having an opening to the hollow channel;
a distal end having an opening to the hollow channel);
wherein the drill guide is coupled to the rod at an angle;
wherein there is a gap having a dimension G between the end of first end portion and drill guide measured along the bottom of rod;
wherein the outer dimension R of the rod is measured proximate the drill guide; and
wherein the ratio of R to D is between 0.2 to 1.0.

2. The drill guide device of claim 1, wherein the ratio of R/D is between 0.7 to 0.9, the ratio of H/R is between 1.5 to 1.7, and the ratio of D/G is between 1.2 to 1.3.

3. The drill guide device of claim 1, wherein the ratio of R/D is between 0.6 to 1.0, the ratio of H/R is between 1.3 to 1.9, and the ratio of D/G is between 1.0 to 2.0.

4. The drill guide device of claim 1, wherein the ratio of R/D is between 0.4 to 1.0, the ratio of H/R is between 1.0 to 2.0, and the ratio of D/G is between 0.1 to 10.0.

5. The drill guide device of claim 1, wherein the ratio of R/D is about 0.8; the ratio of H/R is about 1.6; the ratio of D/G is about 1.25.

6. The drill guide device of claim 1, wherein the ratio of R/D is between 0.4 to 0.6, the ratio of H/R is between 1.5 to 1.7, and the ratio of D/G is between 1.5 to 2.5.

7. The drill guide device of claim 1, wherein the ratio of R/D is between 0.3 to 0.7, the ratio of H/R is between 1.3 to 1.9, and the ratio of D/G is between 1.0 to 5.0.

8. The drill guide device of claim 1, wherein the ratio of R/D is between 0.2 to 1.0, the ratio of H/R is between 1.0 to 2.0, and the ratio of D/G is between 0.1 to 10.0.

9. The drill guide device of claim 1, wherein the ratio of R/D is about 0.5; the ratio of H/R is about 1.6; the ratio of D/G is about 2.0.

10. The drill guide device of claim 1, wherein the ratio of R/D is between 0.4 to 1.0, the ratio of H/R is between 1.0 to 8.0, and the ratio of D/G is between 0.1 to 10.0.

11. The drill guide device of claim 1, wherein the angle between the longitudinal handle axis (h) and the drill guide axis (d) is an obtuse angle.

12. The drill guide device of claim 1, wherein the first end portion (143) of the hand grip (140) is chamfered.

13. The drill guide device of claim 1, further comprising:
a second drill guide extending along a longitudinal drill guide axis (d) and forming a hollow channel configured and adapted to receive a drill bit, the second drill guide having an outer dimension D′ as viewed from the top of the handle, the second drill guide comprising:
a proximal end having an opening to the hollow channel;
a distal end having an opening to the hollow channel;
wherein the second drill guide is coupled to the rod at an angle and at an opposite end of the rod than the second drill guide;
wherein the hand grip has an outer dimension H′ at the second end portion;
wherein an outer dimension R′ of the rod is measured proximate the second drill guide;
wherein there is a second gap having a dimension G′ between the end of second end portion and second drill guide measured along the bottom of rod; and
wherein the ratio of R′ to D′ is between 0.2 to 1.0.

14. The drill guide device of claim 13, wherein the ratio of R′/D′ is between 0.7 to 0.9, the ratio of H′/R′ is between 1.5 to 1.7, and the ratio of D′/G′ is between 1.2 to 1.3.

15. The drill guide device of claim 13, wherein the ratio of R′/D′ is between 0.6 to 1.0, the ratio of H′/R′ is between 1.3 to 1.9, and the ratio of D′/G′ is between 1.0 to 2.0.

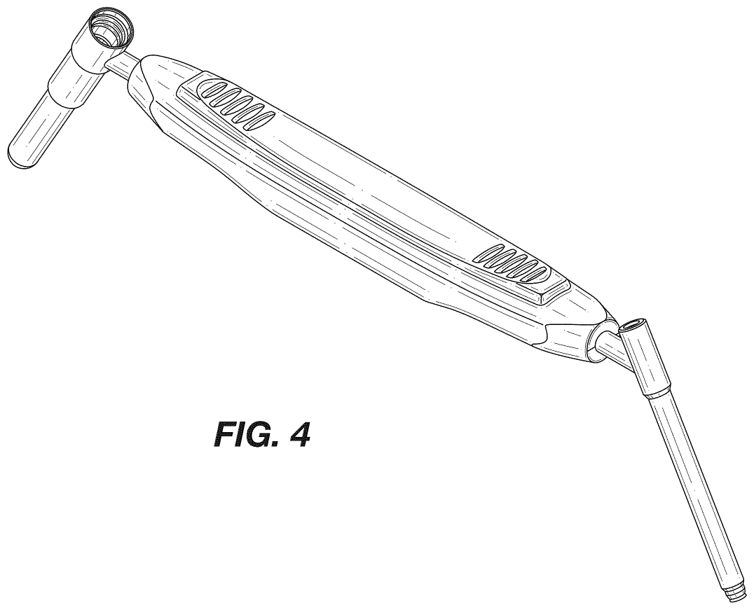

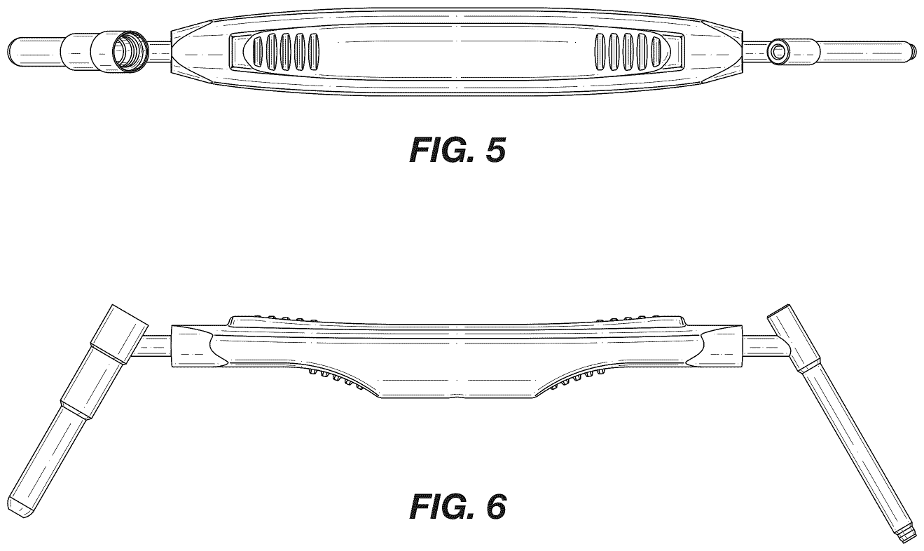

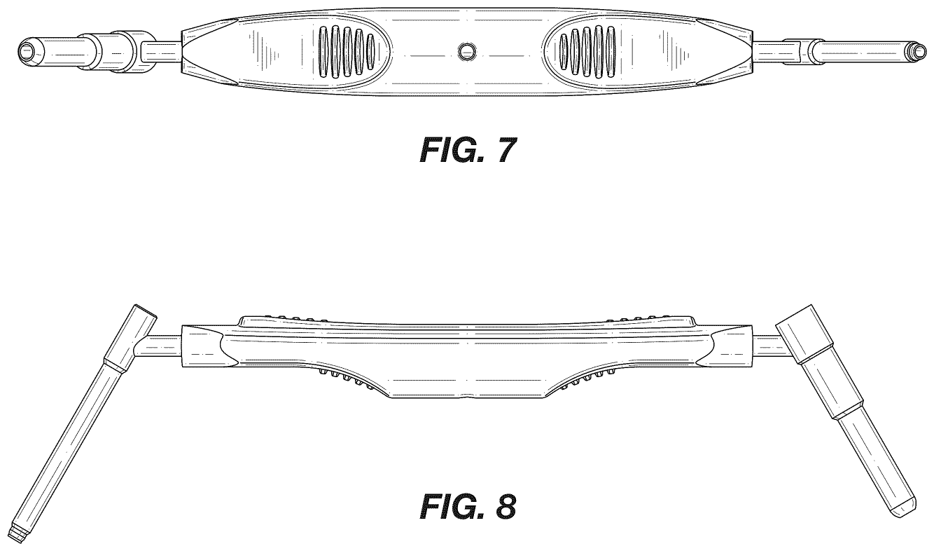

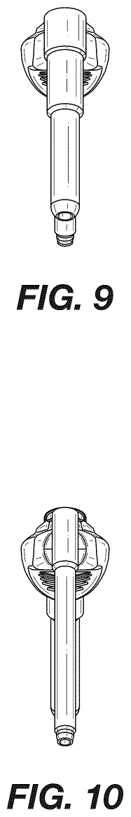

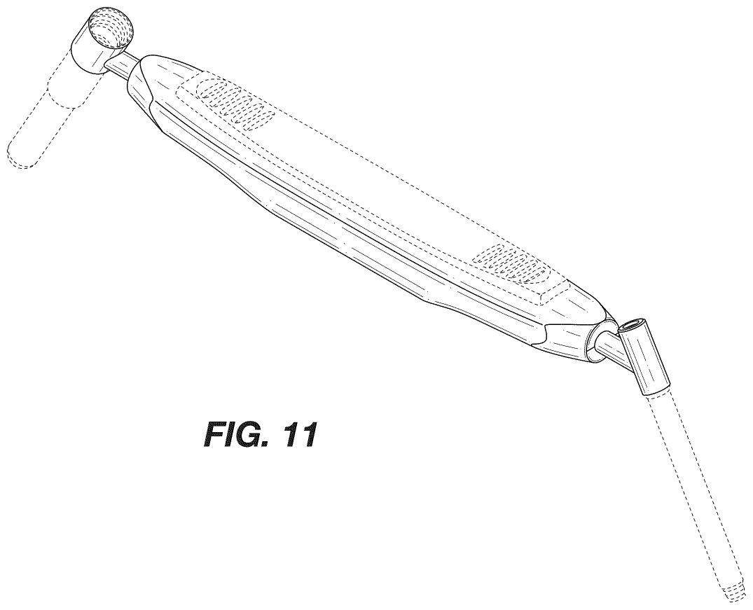

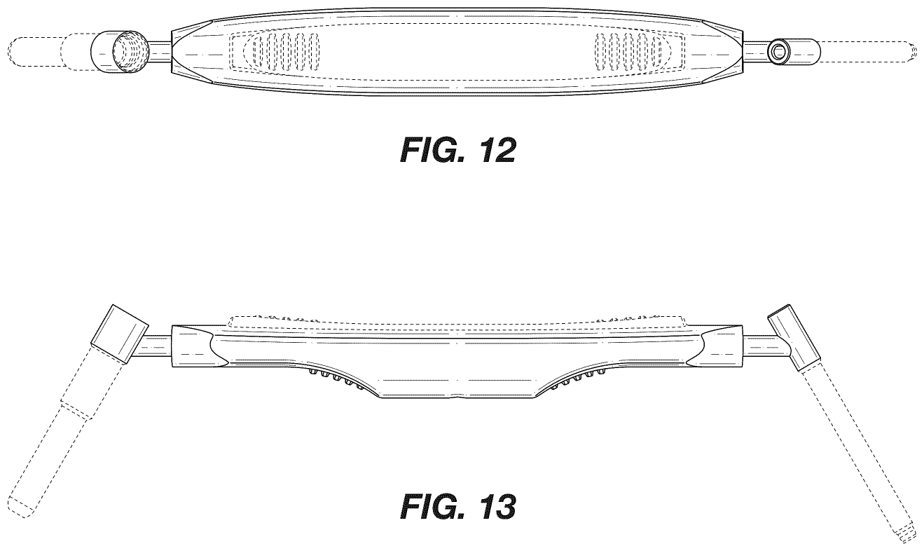

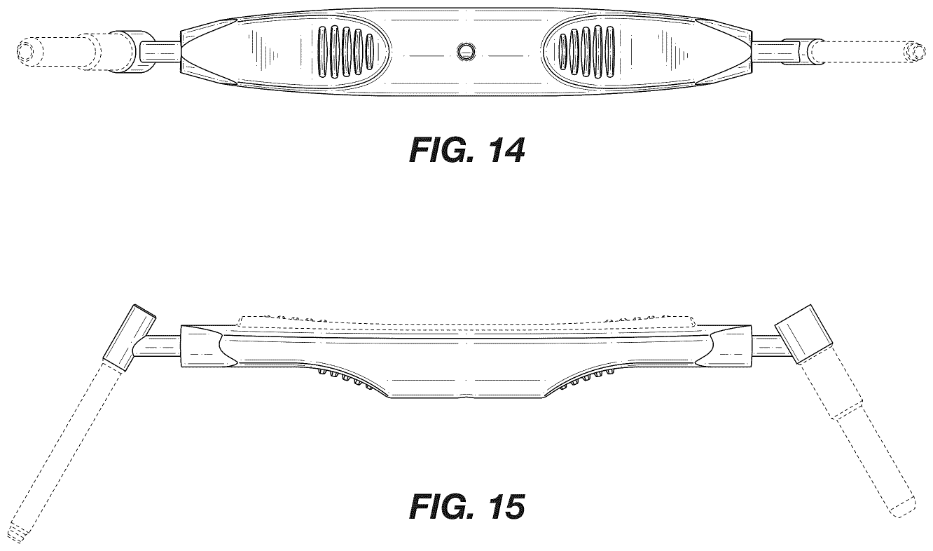

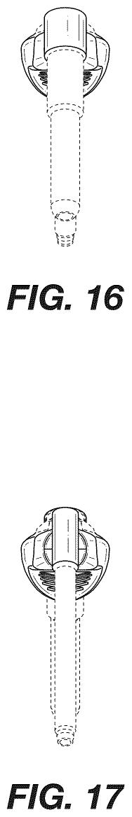

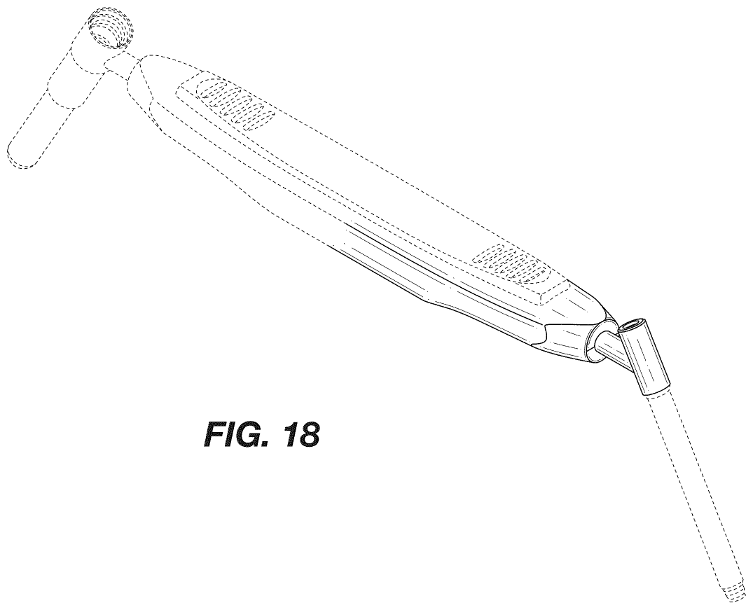

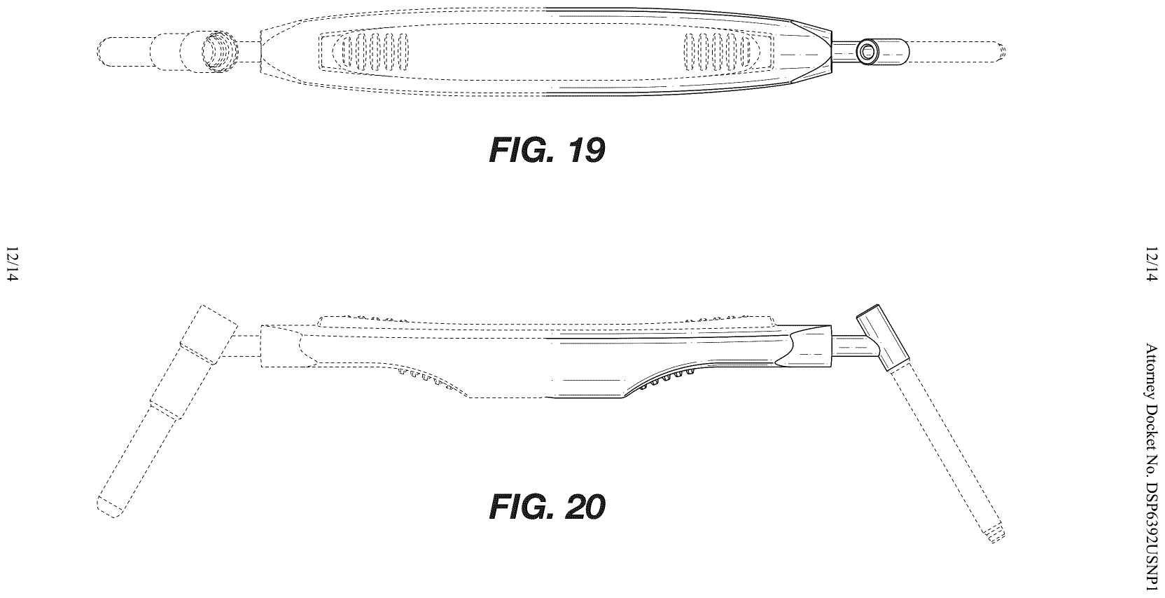

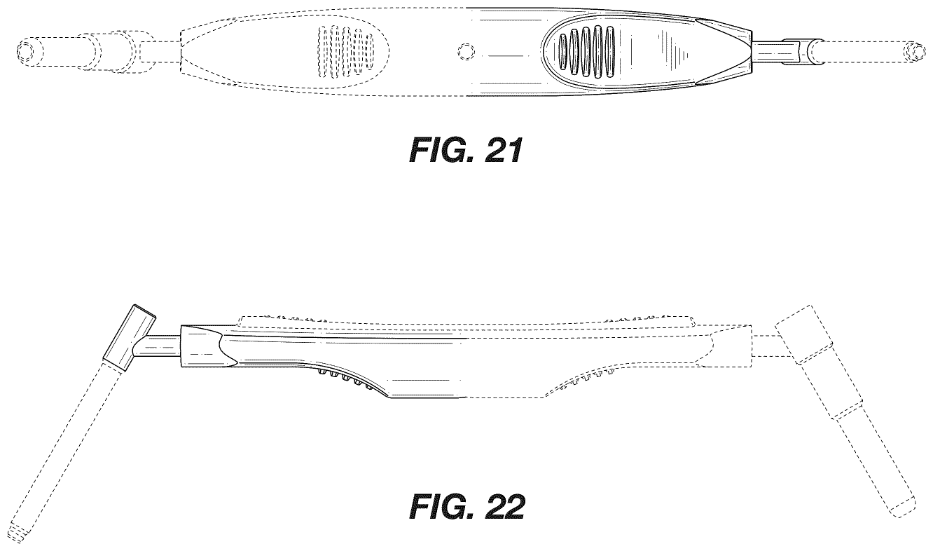

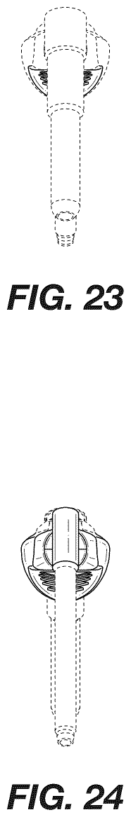

16. The drill guide device of claim 13, wherein the ratio of R′/D′ is between 0.4 to 1.0, the ratio of H′/R′ is between 1.0 to 2.0, and the ratio of D′/G′ is between 0.1 to 10.0.
What is further claimed is the ornamental design for drill guide device, as shown and described in FIGS. 4-10. The broken lines immediately adjacent to the shaded areas depict the bounds of the claimed invention, while all other broken lines are directed to the environment. The broken lines form no part of the claimed invention.
What is further claimed is the ornamental design for drill guide device, as shown and described in FIGS. 11-17. The broken lines immediately adjacent to the shaded areas depict the bounds of the claimed invention, while all other broken lines are directed to the environment. The broken lines form no part of the claimed invention.
What is further claimed is the ornamental design for drill guide device, as shown and described in FIGS. 18-24. The broken lines immediately adjacent to the shaded areas depict the bounds of the claimed invention, while all other broken lines are directed to the environment. The broken lines form no part of the claimed invention.
